(19) United States
(12) Patent Application Publication
(10) Pub. No.: US 2015/0112146 A1
(43) Pub. Date: Apr. 23, 2015

(54) Video Laryngoscope with Adjustable Handle Mounted Monitor

(71) Applicant: Jill Donaldson, Indianapolis, IN (US)

(72) Inventor: Jill Donaldson, Indianapolis, IN (US)

(21) Appl. No.: 14/493,655

(22) Filed: Sep. 23, 2014

Related U.S. Application Data
(60) Provisional application No. 61/893,511, filed on Oct. 21, 2013.

(51) Int. Cl. A61B 1/00; A61M 16/04; A61B 1/07; A61B 1/06; A61B 1/267; A61B 1/05

(52) U.S. Cl. CPC A61B 1/00048; A61B 1/267; A61B 1/00052; A61B 1/051; A61B 1/00066; A61B 1/00032; A61B 1/00117; A61B 1/07; A61B 1/0676; A61M 16/0488
USPC 600/188

(57) ABSTRACT

Provided is a video laryngoscope having a repositionable display screen. The screen is secured via a flexible arm to the outermost area of the handle member second end. This flexible arm may be flexible metal conduit to enable support of the display screen and manipulation of position. A plurality of blade members of differing sizes and material construction may secure individually to the end of the handle member opposing the flexible arm. The blade members are removable. A length of fiber optic cable runs from the display screen, through the handle member and is inserted through an aperture in the blade member. The lens of a fiber optic camera, and a light source are disposed at a free end of the cable to provide video feedback of the environment near the working end of the blade member during a laryngoscopy procedure.

### CROSS REFERENCE TO RELATED APPLICATION

This application claims the benefit of U.S. Provisional Application No. 61/893,511 filed on Oct. 21, 2013 entitled “Laryngoscope with Camera and Handle Monitor.” The above identified patent application is herein incorporated by reference in its entirety to provide continuity of disclosure.

### BACKGROUND OF THE INVENTION

1. Field of the Invention

The present invention relates to medical devices. More particularly, it relates to a medical device used to aid in intubating a patient. The device is a laryngoscope with a flexible camera at the working end to visualize or record footage of the patient's anatomy. A small display is mounted to the handle and is connected to the camera to enable the medical professional to monitor the progress of intubation.

Endotracheal intubation (intubation) involves the insertion of flexible tubing into the trachea to establish an artificial air pathway, or provide a conduit for the administration of therapeutic agents. The procedure is generally performed on seriously injured, ill, or anesthetized patients to aid in ventilation and respiration of the lungs, as well as reducing the risk of airway obstruction. This can also include mechanical or machine-aided ventilation, such as artificial respirators.

There are two primary forms of tracheal intubation. Nasotracheal intubation involves the insertion of tubing through the nasal opening and cavity, down into the trachea. Orotracheal intubation is the insertion of tubing through the oral cavity down into the trachea. Both methods are generally performed after administration of anesthetic, either general or localized. Specific equipment used depends upon the method of intubation, but a laryngoscope or a video laryngoscope is the most common aids.

A laryngoscope is a surgical device having a handle and several interchangeable blades. The handle generally houses a power source (batteries or a plug) and an illuminator. Illuminators enable the medical professional to see into the tracheal pathway, thereby improving tubing placement and reducing the risk of injury caused by blind intubation. Blades come in both straight and curved designs. Use of one design depends upon individual patient anatomy and physician preference.

Video laryngoscopes include fiber optic image visualizing and recording cameras disposed at the working end of the handle or the blade. These devices have grown in popularity over the last two decades because they provide medical professionals with a view of the patient's tracheal anatomy. Images may be displayed on a monitor directly connected to the device, or to a separate display operatively connected to the laryngoscope. Such devices further reduce the risk of injury to a patient's soft tissue, by helping the physician steer the tubing to avoid injured areas. Even with this increased benefit, these devices are still problematic because they require the physician to twist the laryngoscope at an odd angle, or to look up at a separate monitor to check progress. They do not allow a physician to continue the procedure while checking the display device.

A video laryngoscope is needed that provides a physician with the ability to view blade position within the patient anatomy, without pausing the procedure, or directing their attention away from the patient. The present invention provides a laryngoscope with a video monitor mounted to an arm extending outward from the distal end of the handle. The display is tilted at an angle that renders it highly visible to an intubating physician.

2. Description of the Prior Art

Devices have been disclosed in the prior art that relate to laryngoscopes. These include devices that have been patented and published in patent application publications. These devices generally relate to laryngoscopes with monitoring means. The following is a list of devices deemed most relevant to the present disclosure, which are herein described for the purposes of highlighting and differentiating the unique aspects of the present invention, and further highlighting the drawbacks existing in the prior art.

Laryngoscopes come in a variety of shapes and configurations. In general, each laryngoscope features a blade member and a handle used to manipulate the position of the blade member. Older models feature straight blades. While newer devices may have curved blades that better conform to the shape of human tracheal passages. One example of a laryngoscope is disclosed in Mcgrath, U.S. patent application publication no. 2010/0312059. Another example is disclosed in Hakanen, U.S. Pat. No. 8,414,481.

Intubation of patients requires the delicate insertion of a tube into the pharyngeal pathway of a human throat. The medical professional must hold the pathway open with a laryngoscope while feeding the tube through the opening. To make this task easier, laryngoscopes have been developed that include cameras mounted near or at their working end. Cameras record images or data associated with the surrounding anatomy and transmit this information back to a processing unit and coupled display. These features allow physicians to obtain real-time information about the intubation procedure.

Berall, U.S. Pat. No. 5,827,178 discloses a laryngoscope with a camera mounted on the working end of the blade. The laryngoscope has a handle connected to a blade member at approximately a ninety degree angle. A fiber optic camera is disposed at the working end of the blade. This camera is connected to a screen mounted on the handle by a fiber optic cable running through the device. In this way, a physician can easily view the environment within a patient's throat while intubating. Unlike the present invention the Berall device does not have a removable and manipulable blade. Nor does Berall have a display flexibly mounted to one end of the handle at an upward angle for easy viewing.

A similar device is disclosed by Mcgrath, U.S. patent application publication no. 2013/0057667. The Mcgrath device differs from the Berall device in that it has a curved blade and that the display screen extends laterally from the distal portion of the handle rather than a proximal portion. Another laryngoscope with integrated camera element is disclosed in Mcgrath U.S. patent no. 2007/0167686. This patent teaches a laryngoscope with a disposable blade that has a channel running therethrough. The channel holds a camera cable and guides the camera through the blade and body. It does not include an attached display unit. Neither of the Mcgrath inventions discloses a laryngoscope with a display screen disposed on the distal end of the handle like that of the present invention.

Pecherer, U.S. Pat. No. 8,251,898 discloses a laryngoscope with a hollow blade. The blade has deflectors such as mirrors located inside it's hollow interior and a lens disposed at the upper end of the blade. In use, the physician looks down through the lens to view the deflected image. Unlike the present invention the Pecherer device requires users to look down over the blade rather than viewing an upwardly angled display screen attached to the handle. The present invention provides improved ability to maneuver the device while still viewing the target anatomy, because it does not require the user to hover over the top of the blade, and the patient's throat, in order to see what is going on.

Finally, Miller, U.S. patent application publication no. 20070179342 teaches a wireless laryngoscope with integrated camera. The blade is two separate portions secured together to form a hollow interior region that houses a camera element. A light may be incorporated into the end of the blade assembly to provide illumination to a target area of the patient's anatomy. Power source, processing unit, and a transmitter are disposed within the handle of the device. Video captured via the camera is transmitted to a remote receiver using the transmitter. In this way, the physician can view patient anatomy on a large screen. Additionally, the remote receiver may be a large screen display suitable for viewing by a group of students or onlookers. The Miller device does not have a conveniently located display screen attached to the distal end of the handle. The present invention provides such a screen in order to aid physicians during intubation.

These prior art devices have several known drawbacks. They do not disclose a display screen disposed on a flexible and repositionable arm at the end of the handle. Nor do the devices disclose a blade that is removable for easy cleaning. The present invention provides these features. It substantially diverges in design elements from the prior art and consequently, it is clear that there is a need in the art for an improvement to existing laryngoscope devices. In this regard the present invention substantially fulfills these needs.

### SUMMARY OF THE INVENTION

In view of the foregoing disadvantages inherent in the known types of video laryngoscope now present in the prior art, the present invention provides a new easily visible display unit wherein the same can be utilized for providing increased patient safety and added convenience for the user when checking on the progress of intubation during a procedure.

The present invention is a video laryngoscope with a repositionable display screen. The device comprises a handle member and one or more removable blades that removably secure to the handle member, and a display screen mounted to the handle member via a flexible arm. Blades are interchangeably secured to the handle member, enabling a physician to switch out blades according to patient anatomy, without having to have multiple laryngoscopes. This feature provides modular flexibility to the device and reduces cost, as well as the number of laryngoscopes a facility must own.

A fiber optic cable extends from the display screen, through the flexible arm, through the handle member, and protrudes from the working end of the handle member. Each blade member has a channel or tunnel extending from the upper end of the blade to the working end or an area near the working end. The fiber optic cable is fed through this tunnel in order to secure it in place during an intubation proceeding. When correctly positioned the cable concludes near the working end of the blade. A camera and light source are integrated into the free end of the fiber optic cable, for visualizing and/or capturing video of the anatomy surrounding the blade. Captured data is relayed to the display screen, where it is visually output.

During an intubation procedure, the physician holds the device by the handle and manipulates it in different directions to open the tracheal passages. The procedure can involve a great deal of twisting and rotation of the laryngoscope. To ensure that the physician is always able to obtain an easily viewable image of the patient anatomy, the display screen is attached to the handle member via a flexible arm. The physician can use his or her free hand to bend and manipulate the display screen into a desired position. In this way, the medical professional can maintain constant supervision over the tracheal passages.

It is therefore an object of the present invention to provide a new and improved video laryngoscope device that has all of the advantages of the prior art and none of the disadvantages.

It is another object of the present invention to provide a video laryngoscope with a freely repositionable display screen in connection with a camera positioned at the laryngoscope blade working end.

Another object of the present invention is to provide a means for enabling physicians intubating a patient to monitor the anatomical environment surrounding a laryngoscope blade.

Yet another object of the present invention is to provide a video laryngoscope with interchangeable blades in varied sizes to accommodate patients of different sizes.

Still another object of the present invention is to provide a video laryngoscope with a repositionable display screen such that the device may be used by both right and left handed physicians.

A further object of the present invention is to provide a video laryngoscope that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 provides a perspective view of the video laryngoscope in use during a patient intubation.

FIG. 2 shows a perspective view of the disassembled video laryngoscope.

FIG. 3 shows a perspective view of the assembled video laryngoscope.

### DETAILED DESCRIPTION OF THE INVENTION

Reference is made herein to the attached drawings. Like reference numerals are used throughout the drawings to depict like or similar elements of the video laryngoscope. For the purposes of presenting a brief and clear description of the present invention, the preferred embodiment will be discussed as used for aiding in intubation of patients. The figures are intended for representative purposes only and should not be considered to be limiting in any respect.

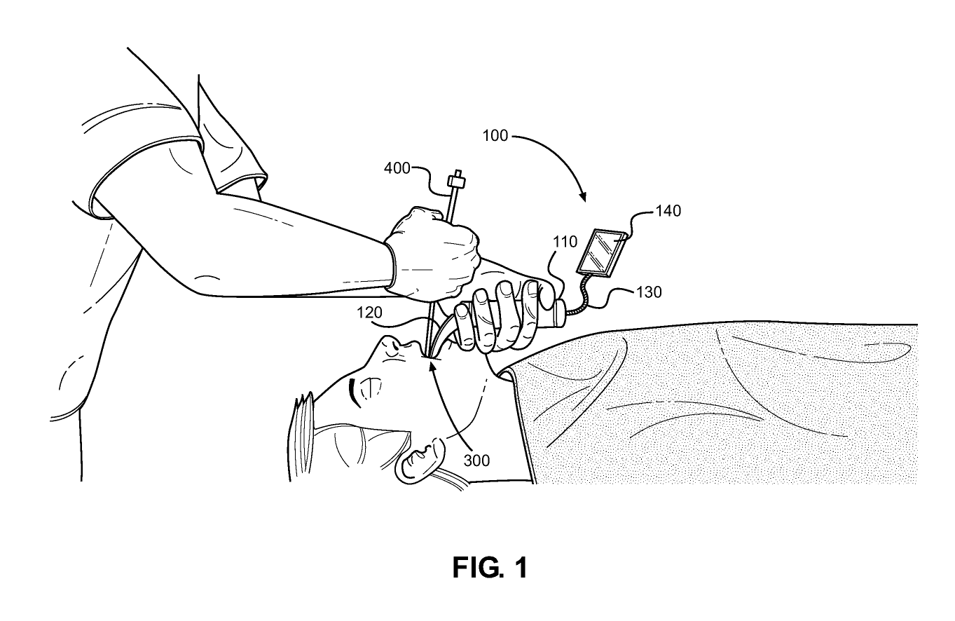

Referring now to FIG. 1, there is shown a video laryngoscope according to the present invention, in use. A physician grips the laryngoscope 100 by the handle member 110. One of a plurality of interchangeable blade members 120 is removably secured to a first end of the handle member. The blade is inserted into the patient's oral cavity 300, where it holds the tracheal passages open to permit insertion of a length of tubing 400. Blade members may be curved, as shown, or straight, to accommodate different intubation procedure techniques. The blade members may be constructed of reusable materials such as metals, or alternatively may be constructed of disposable plastics.

Attached to the second end of the handle member 110 is a flexible arm 130, which physically and operatively connects to a display screen 140. The flexible arm can be bent and twisted into different configurations, according to the desired positioning of the display screen. A camera and light source positioned at the working end of a blade member (not shown) extends through same, through the handle member and flexible arm, and ultimately connects to the display screen. This camera captures video data of the area surrounding the blade member working end transmits it to the display screen. Medical professionals can monitor the progress of the procedure via the display screen. Because the display screen support is a flexible arm, it can be repositioned as needed, to provide the user with an upright view of the on-screen image.

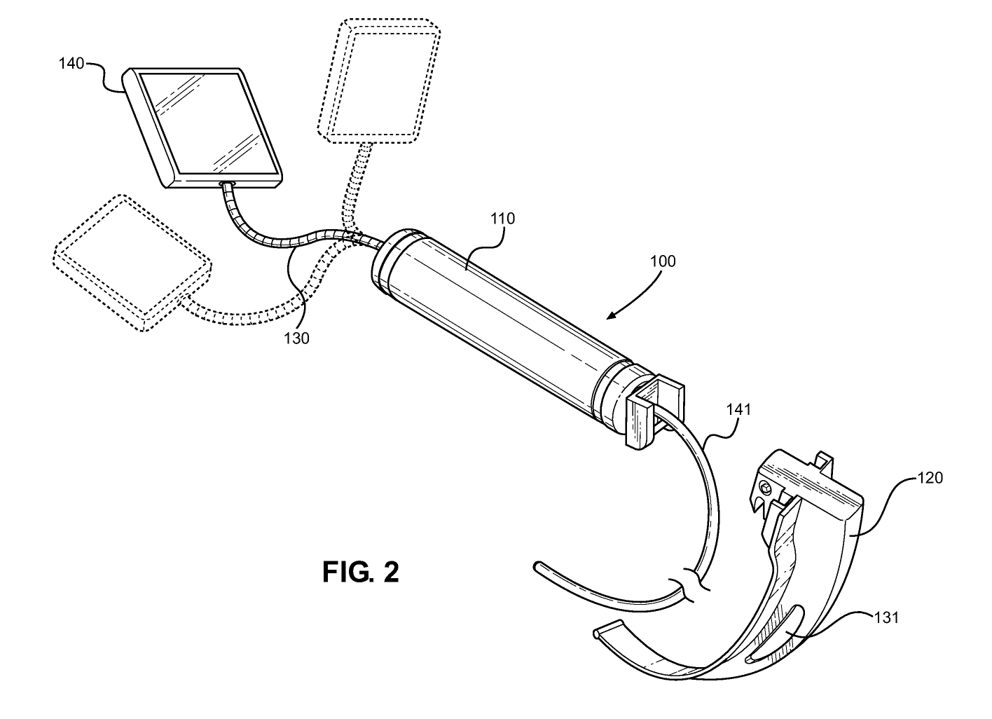

Turning now to FIG. 2, the video laryngoscope is shown in a disassembled state. The laryngoscope 100 has an elongated handle member 110, which is depicted in the images as having a cylindrical shape. This is for illustrative purposes only, as the handle member may have a square, octagonal, or other suitable geometric cross-section. It may also have a molded grip, or may be coated or covered in a material having a high coefficient of friction in order to reduce the risk that the operator's hand will slip during an intubation procedure.

Blade members 120 removably secure to the first end of the handle member 110. In the depicted example, the blade member has a hook shaped catch that engages with a slot disposed on the first end of the handle member. Such a configuration enables firm retention of the blade member during an intubation procedure, while allowing a medical professional to switch out blade members as necessary. Numerous alternative attachment techniques are known in the laryngoscope art. By way of example, an alternative securing configuration includes a cuff attached to the blade member, which overlaps and envelops a portion of the handle when in position.

The blade member 120 is separated from the handle member 110 exposing the fiber optic cable 141. This cable has an integrated camera 140 at its free end. The remaining portion of the cable extends through the handle member, and flexible arm, and connects to the display screen. It should be understood that while the cable is described as fiber optic, other forms of video data transfer connectors might be substituted. The cable should be flexible and lightweight, to enable easy insertion into the blade member during use.

A flexible arm 130 secures the handle member 110 to the display screen 140. The arm is preferably a length of conduit that allows articulation in multiple directions, but is still sufficiently strong to main the display screen in position until force is applied to the screen or arm. By way of example, flexible steel conduit or other “snake” style conduit may be used to house the fiber optic cable and support the display screen. The flexible arm secures at a first end to the second end of the handle member in a parallel or in-line configuration such that the flexible arm protrudes outward along the same axis as the length of the handle member. The second end of the flexible arm secures to the display screen. Point of securement between the display screen and flexible arm can vary depending on the weight and type of display screen. The display screen itself may be any suitable display such as LCD, or the like. Electrical and processing components necessary to convert video data captured by a fiber optic camera into on-screen visual output are known to those in the art of medical device engineering, therefore such details will not be discussed herein.

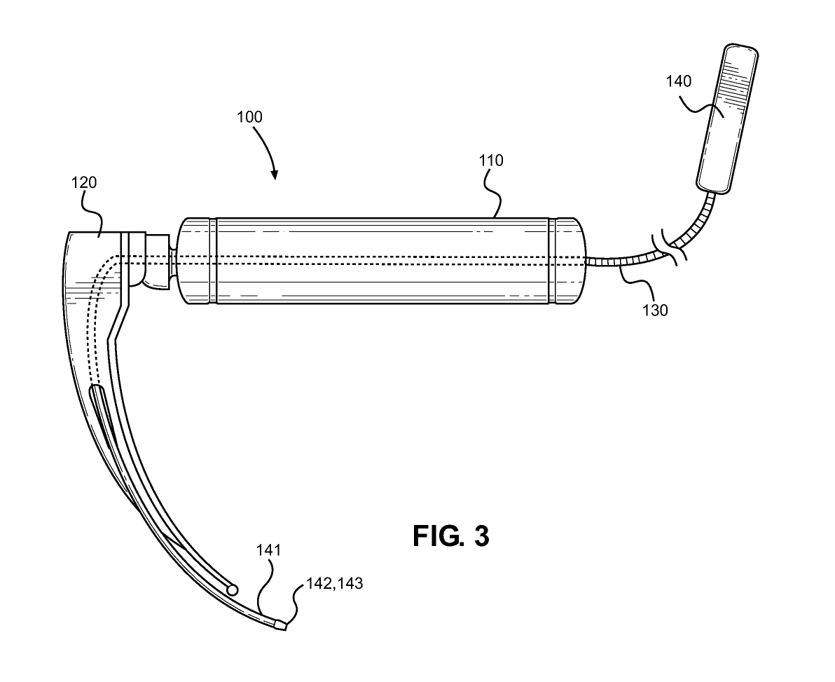

Referring now to FIG. 3, the video laryngoscope is shown in a ready for use state. The fiber optic cable 141 is inserted through an opening 131 at the upper end of the blade member 120 (shown in FIG. 2). This opening provides access to a tunnel or channel running through the blade member and exiting near the working end thereof. To prep the device for use, a physician gently threads the free end of the cable through the opening and applies continuous or repeated force until the cable exits the other side of the tunnel. Optionally, a u-shaped channel may run along the forward lower portion of the blade to provide a guide or retaining area for the free end of the cable. Once the cable is in position, the blade is secured to the handle member 110. The camera 142, and light source 143 should lie proximal to the working end of the blade member but does not extend past same. In the depicted illustration, the cable 140 extends past the tip of the blade member working end in order to demonstrate the position of the light source and camera, but in practice, the cable should not protrude past the working end of the blade member.

A power source is housed within the handle member 110. The power source is one or more batteries. These batteries may be disposable, thereby necessitating an access door or hatch in the handle member. Access doors such as slideably removable doors are known in the art and can be constructed in a variety of configurations. Additionally the batteries may be rechargeable. In such an embodiment, the handle member may further comprise a dc power port, a usb port, or the like.

The present invention is a video laryngoscope with a manipulable and repositionable display screen. The device enables physicians to monitor the progress of an intubation procedure without having to twist or reposition the laryngoscope itself. In this way, the device reduces the potential for injury resulting from wiggling of a laryngoscope and detecting the potential for an undesirable esophageal inhibitor during an intubation procedure.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A laryngoscope, comprising:
a handle member having a first end and a second end;
a plurality of blade members, wherein each of said blade members has a working end and an upper end, and wherein each of said blade members has a tunnel running from an aperture in said upper end to an aperture disposed near said working end;
a flexible arm attached to and protruding outward from said second end of said handle member;
a display attached to a distal end of said flexible arm;
a camera operatively connected to said display screen via a cable, wherein said cable extends through said flexible arm and said handle member, and is removably threadable through said tunnel in each of said blade members;
a power source disposed within said handle member.

2. The laryngoscope of claim 1, wherein said flexible arm protrudes outward from said second end of said handle member along the same axis as the length of said handle member.

3. The laryngoscope of claim 1, wherein said blade members are curved.

4. The laryngoscope of claim 1, wherein said power source is batteries.

5. The laryngoscope of claim 4, wherein said batteries are rechargeable.

6. The laryngoscope of claim 1, wherein said display screen is adapted to display video playback captured by said camera.

7. The laryngoscope of claim 1, wherein said flexible arm is capable of supporting said display screen.

8. The laryngoscope of claim 1, wherein said flexible arm is freely repositionable thereby enabling manipulation of the orientation and position of said display screen.

9. The laryngoscope of claim 1, wherein said cable is a fiber optic cable.

10. The laryngoscope of claim 1, wherein said plurality of blade members includes blade members having different shapes and or sizing.

11. The laryngoscope of claim 1, further comprising:
a light source incorporated into said cable and disposed proximal to said camera.
